(19) United States
(12) Patent Application Publication
(10) Pub. No.: US 2009/0241963 A1
(43) Pub. Date: Oct. 1, 2009

(54) FLUID-FLOW CONNECTORS AND ARRAGEMENTS

(75) Inventor: Nicholas Paul MacMillan, Temple Ewell (GB)

Correspondence Address: LOUIS WOO;LAW OFFICE OF LOUIS WOO, 717 NORTH FAYETTE STREET, ALEXANDRIA, VA, 22314, US

(73) Assignee: Smiths Group plc, London (GB)

(21) Appl. No.: 12/310,029

(22) PCT Filed: Aug. 13, 2007
(86) PCT No.: PCT/GB2007/003089

§ 371 Date: Feb. 6, 2009

(30) Foreign Application Priority Data
Aug. 23, 2006 (GB) 0616687.0

(51) Int. Cl. A61M 16/04; A61M 1/00

(52) U.S. Cl. 128/207.14

(57) ABSTRACT

A suction arrangement includes a suction source (1) with a single inlet (13) and a connector (5) of caltrop shape with four interconnected ribbed ports 50 to 53, one of which (50) is connected to the suction inlet. The other three ports (51 to 53) are connected with tubing interconnections (15, 23, 33, 46) which each have a valve or the like (25, 35, 48) for blocking flow as necessary. The tubing interconnections connect with respective suction appliances including a suction outlet (41) above the cuff (42) of a tracheal tube (40), a tracheal suction catheter (3) and an oral suctioning catheter (2).

This invention relates to fluid-flow connectors.

The invention is more particularly concerned with arrangements for medico-surgical applications, such as suctioning, irrigation and medication delivery.

Many surgical procedures require suctioning, to remove blood, secretions, tissue debris and the like. A typical hospital intensive therapy unit (ITU) uses suctioning for three different purposes: ventilation suctioning (such as to remove secretions that accumulate in the respiratory passages), subglottic suctioning (to remove secretions that collect above the sealing cuff of a tracheal tube) and oral suctioning. Most therapy locations in an ITU are only provided with a single suction outlet. The usual practice is to use a Y-connector to enable two suction apparatus to be connected to a common outlet. The disadvantage of this is that the clinician is forced to disconnect one of the suction apparatus if he needs to use an alternative or additional suction apparatus. This can be time consuming and messy with a higher risk of cross contamination As a result of this the clinician may avoid or delay certain vital cleaning procedures and consequently put the recovery of the patient at risk. It is also often necessary to provide multiple fluid outlets for irrigation purposes or for delivery of medication to different sites.

It is an object of the present invention to provide alternative fluid-flow apparatus and arrangements.

According to one aspect of the present invention there is provided a fluid-flow connector of the above-specified kind, characterised in that the connector includes four interconnected ports arranged for connection respectively to a source and to three fluid-flow appliances.

The source may be a suction source or a source of irrigation liquid or medication. The ports are preferably ribbed externally to retain tubing pushed onto the port. The connector preferably has a caltrop shape with the ends of the ports arranged at the apices of a tetrahedron. At least one of the ports may have associated therewith a tethered cap by which the port can be closed when not needed.

According to another aspect of the present invention there is provided a fluid-flow arrangement including a source, a connector according to the above one aspect of the present invention, a first tubing interconnection extending between the source and a first port of the connector, three fluid-flow appliances, and three further tubing interconnections connected between the fluid-flow appliances and respective ones of the other ports of the connector.

One or more of the tubing interconnections may include an arrangement for blocking flow through the tubing interconnections. The source may be a suction source and at least one of the appliances is preferably selected from a group consisting of a suction outlet above the cuff of a tracheal tube, a tracheal suction catheter and an oral suctioning catheter. The source may be a suction source and the appliances may include a suction outlet above the cuff of a tracheal tube, a tracheal suction catheter and an oral suctioning catheter.

A suction fluid-flow arrangement including a suction connector, according to the present invention, will now be described, by way of example, with reference to the accompanying drawings, in which:

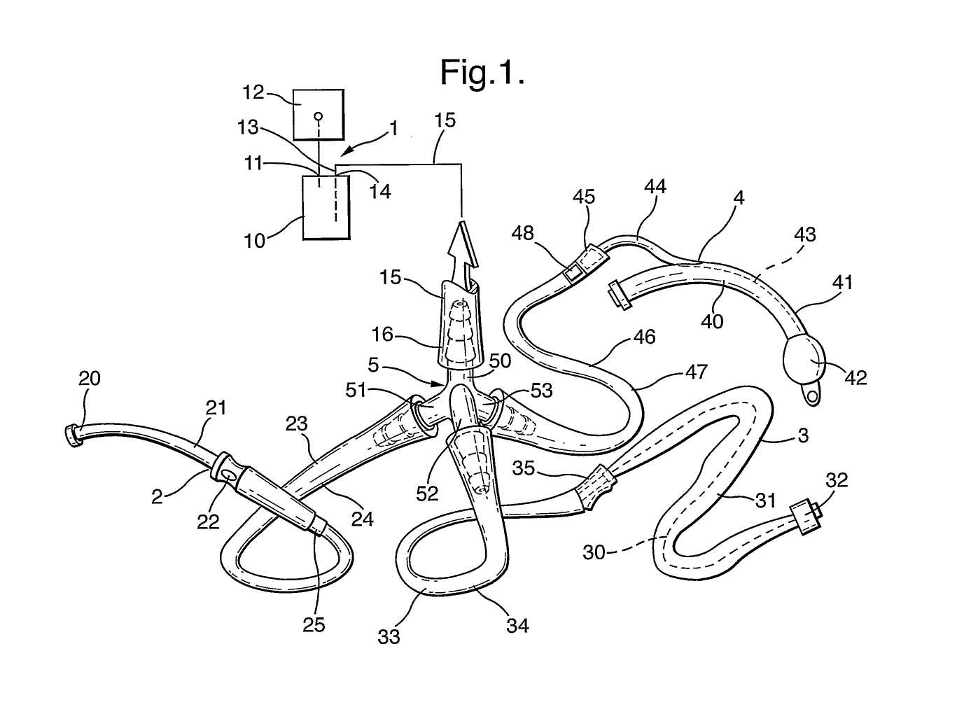

FIG. 1 is a perspective view of the arrangement;

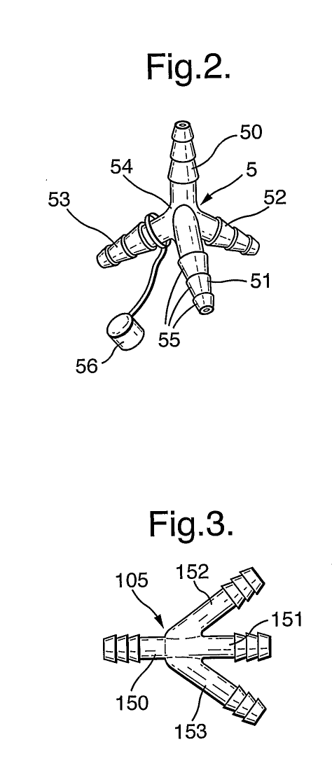

FIG. 2 is a perspective view of the connector without tubing attached; and

FIG. 3 is a plan view of an alternative connector.

With reference first to FIGS. 1 and 2, the suction arrangement includes a source of suction 1 connected with three different suction appliances 2 to 4 via a four-way connector 5.

The suction source 1 is a conventional suction container 10 having an outlet 11 connected with a wall-mounted suction outlet 12, and an inlet 13 connected to one end 14 of a length of flexible tubing 15. When turned on, the suction outlet 12 draws material from the tubing 15 into the container 10 where it is retained for disposal. The opposite end 16 of the tubing 15 is fitted onto the outlet port 50 of the connector 5.

The connector 5 is moulded of a rigid plastics material and has four hollow arms or ports 50 to 53 extending in a star configuration in three dimensions from a central hub 54 and inclined at 120° to one another. The connector 5, therefore, has a symmetrical, caltrop or jack shape with the free ends of the arms being located at the apices of a tetrahedron. Each arm 50 to 53 is formed with ribs 55 towards its outer end, the ribs being tapered to an increased diameter inwardly so as to enable tubing to be pushed onto the arms relatively easily but to resist removal of the tubing by inadvertent pulling. It will be appreciated that each of the four port 50 to 53 communicates with each of the other ports via the hub 54. One or more of the ports are preferably provided with a tethered cap, only one of which 56 is shown. This is used to close the port 53 when not in use, such as when only two suction appliances are connected to the suction source, to prevent loss of suction power.

The first suction appliance 2 is a conventional Yankauer oral suctioning catheter adapted for suctioning within the mouth of the patient. The catheter has a curved forward, patient end 20 and a straight handle 21 at its opposite end, which may have a suction control vent 22 to enable the user to control the amount of suctioning applied. The rear end of the catheter 2 is connected to a tubing interconnection 23 in the form of a length of flexible, resilient tubing 24 and an optional isolation switch 25 adjacent the catheter by which flow from the catheter can be selectively blocked. The other end of the tubing 24 is push fitted over an inlet port 51 of the connector 5.

The second suction appliance 3 is a conventional closed system suction catheter assembly of the kind comprising a suction catheter 30 enclosed within a protective envelope 31 and extensible through a T-piece fitment 32, which can be coupled to the end of a tracheal tube. Closed system suction catheters are sold, for example, by Smiths Medical under the “Stericath” trade mark and by Tyco Medical under the trade mark “Trachcare”. The suction catheter assembly 3 is connected with a second inlet port 52 by means of a second tubing interconnection 33. This tubing interconnection 33 comprises a length of flexible tubing 34, having one end pushed over the connector inlet port 52. An isolation switch 35, by which flow from the catheter assembly 3 can be selectively blocked, is formed by a valve at the machine end of the suction catheter 30 itself.

The third suction appliance 4 is for sub-glottic suctioning and is provided by the suction channel of an endotracheal tube 40 comprising a suction outlet 41 just above the sealing cuff 42 of the tube, a suction lumen 43 extending rearwardly from the outlet within the wall of the tube and a small-bore, flexible suction line 44 extending from the suction lumen and terminated by a connector 45. A third tubing interconnection 46 connects the connector 45 with the third inlet port 53 of the connector 5. The third tubing interconnection 46 comprises a length of flexible tubing 47, having one end pushed over the connector inlet port 53, and an isolation switch 48 by which flow from the suction outlet 41 can be selectively blocked.

It can be seen that this arrangement enables three different suction appliances to be connected to the same suction source without the need for repeated connection and disconnection. After use, the connector and the tubing connected with it would be disposed of in an approved manner.

It is not essential for the connector to have the three-dimensional, caltrop shape described above since any other shape with four ports could be used. For example, FIG. 3 shows a flat, two-dimensional connector 105 where the outlet port 150 is arranged in line with one of the inlet ports 151 and where the other two inlet ports 152 and 153 are arranged in the same plane at an angle of 45° to the first inlet port and on opposite sides. Alternatively, the ports could be arranged in a cross-shape with each port at an angle of 90° to the adjacent port. The invention is not confined to use with the suction appliances described above since alternative suction devices could be used. For example, one of the appliances could be a wound suction catheter. Sub-glottic suctioning need not be achieved by a suction lumen incorporated into the endotracheal tube itself but could be by an alternative suction catheter manually inserted down the trachea outside the endotracheal tube until its end lies close to the upper end of the endotracheal tube sealing cuff.

The connector could include isolation valves built into it to close ports when no tubing was connected to it. These could be used instead of the tethered caps.

The invention is not confined to suction arrangements but could be used in other fluid-flow arrangements, such as, for example, for delivering irrigation liquid or liquid medicine to three different sites.

1. A fluid-flow connector characterized in that the connector includes four interconnected ports arranged for connection respectively to a source and to three fluid-flow appliances.

2. A connector according to claim 1, characterized in that the source is a suction source.

3. A connector according to claim 1, characterized in that the source is a source of irrigation liquid or medication.

4. A connector according to claim 1, characterized in that the ports are ribbed externally to retain tubing pushed onto the port.

5. A connector according to claim 1, characterized in that the connector has a caltrop shape with the ends of the ports arranged at the apices of a tetrahedron.

6. A connector according to claim 1, characterized in that at least one of the ports has associated therewith a tethered cap by which the port can be closed when not needed.

7. (canceled)

8. A fluid-flow arrangement according to claim 11, characterized in that one or more of the tubing interconnections includes an arrangement for blocking flow through the tubing interconnections.

9. A fluid-flow arrangement according to claim 11, characterized in that the source is a suction source and that at least one of the appliances is selected from a group consisting of a suction outlet above the cuff of a tracheal tube, a tracheal suction catheter and an oral suctioning catheter.

10. A fluid-flow arrangement according to claim 11, characterized in that the source is a suction source and that the appliances include a suction outlet above the cuff of a tracheal tube, a tracheal suction catheter and an oral suctioning catheter.

11. A fluid-flow arrangement comprising: a source, a connector having a plurality of interconnected ports arranged for connection respectively to at least the source and a plurality of fluid flow appliances, wherein the fluid-flow arrangement further comprises a first tubing interconnection extending between the source and the first port of the connector, three fluid-flow appliances, and three further tubing interconnections connected between the fluid-flow appliances and respective ones of the other ports of the connector.
